(19) United States
(12) Patent Application Publication
(10) Pub. No.: US 2026/0315342 A1
(43) Pub. Date: Oct. 15, 2026

(54) SOLID EMULSIFIER FOR OIL-IN-WATER TYPE EMULSION, PRODUCTION METHOD OF OIL-IN-WATER TYPE EMULSION, OIL-IN-WATER TYPE EMULSION AND COSMETIC COMPOSITION

(71) Applicants: SANYO CHEMICAL INDUSTRIES, LTD., Kyoto (JP); ADMATECHS CO., LTD., Aichi (JP)

(72) Inventors: Kosuke HAMANO, Kyoto (JP); Yasushi NAKAMURA, Kyoto (JP); Yuji ONO, Aichi (JP); Kenta HORI, Aichi (JP); Nao INOUE, Aichi (JP)

(73) Assignees: SANYO CHEMICAL INDUSTRIES, LTD., Kyoto (JP); ADMATECHS CO., LTD., Aichi (JP)

(21) Appl. No.: 19/111,104

(22) PCT Filed: Sep. 13, 2023
(86) PCT No.: PCT/JP2023/033360

§ 371 Date: Mar. 12, 2025

(30) Foreign Application Priority Data
Sep. 15, 2022 (JP) 2022-147390

(51) Int. Cl. A61K 8/25; A61K 8/06

(52) U.S. Cl. CPC A61K 8/25; A61K 8/062; A61K 2800/412; A61K 2800/612; A61K 2800/80

(57) ABSTRACT

The present invention aims to provide a solid emulsifier for oil-in-water emulsions for use in production of an oil-in-water emulsion that has a less squeaky feeling and a good texture when used as a cosmetic, a method for producing an oil-in-water emulsion, and an oil-in-water emulsion. The present invention relates to a solid emulsifier for oil-in-water emulsions, the solid emulsifier comprising metal oxide particles having a volume average particle size of 0.2 to 0.4 μm, a specific surface area of 5 to 20 m²/g, and a hydrophobicity of 5 to 35, with the hydrophobicity being measured by the following method:

<Method of Measuring Hydrophobicity>
(1) 50 mL of ion-exchanged water is placed in a 100 mL beaker;
(2) 0.2 g of the metal oxide particles as a measurement sample is added to the beaker in the step (1), followed by stirring with a PTFE stirring bar with a length of 30 mm and a diameter of 8 mm, wherein a stirring speed is adjusted so that when a stirring state in the beaker is observed from a side of the beaker, a lowest point of a vortex created by the stirring is positioned above the midpoint of a distance from a bottom of the beaker to a liquid surface;
(3) 0.5 mL of methanol is added dropwise using a burette with the stirring continued, the stirring is stopped after 30 seconds from the dropwise addition, and 30 seconds after stopping the stirring, the presence or absence of the metal oxide particles at the liquid surface is visually checked;
(4) the step (3) is repeated until the metal oxide particles can no longer be visually observed at the liquid surface; and
(5) a percentage of a volume (mL) of the methanol dropwise added in the steps (3) and (4) relative to a sum of a volume (50 mL) of the ion-exchanged water and the volume of the methanol dropwise added in the steps (3) and (4) in the beaker is calculated to determine the hydrophobicity.

### TECHNICAL FIELD

The present invention relates to solid emulsifiers for oil-in-water emulsions, methods for producing oil-in-water emulsions, oil-in-water emulsions, and cosmetic compositions.

### BACKGROUND ART

Oil-in-water (O/W) emulsions and water-in-oil (W/O) emulsions are applied to cosmetics and the like. Most of the emulsifiers used in emulsions are surfactants. Cosmetics containing surfactants can be sticky or slimy when applied to the skin, causing an unpleasant texture when used, can cause skin irritation when applied to the skin, and can come off when exposed to water such as sweat, due to issues with water resistance.

In recent years, oil-in-water emulsion cosmetics that are non-irritating to the skin and have good water resistance have been developed (Patent Literature 1). The cosmetics do not contain surfactants and utilize Pickering emulsification with inorganic fine particles having an average particle size of less than 200 nm, such as silica fine particles.

Such cosmetics, however, have issues such as an unpleasant texture caused by squeakiness due to inorganic fine particles.

### CITATION LIST

### Patent Literature

Patent Literature 1: JP 2001-518111 T

### SUMMARY OF INVENTION

### Technical Problem

The present invention aims to provide a solid emulsifier for oil-in-water emulsions for use in production of an oil-in-water emulsion that has a less squeaky feeling and a good texture when used as a cosmetic, a method for producing an oil-in-water emulsion, an oil-in-water emulsion, and a cosmetic composition.

### Solution to Problem

The present inventors have conducted extensive research to solve the above problems and have arrived at the present invention.

Specifically, the present invention relates to a solid emulsifier for oil-in-water emulsions, the solid emulsifier containing metal oxide particles having a volume average particle size of 0.2 to 0.4 μm, a specific surface area of 5 to 20 m²/g, and a hydrophobicity of 5 to 35; also relates to a method for producing an oil-in-water emulsion containing an aqueous phase component and an oil phase component dispersed in the aqueous phase component, the method including a dispersion step including dispersing the solid emulsifier for oil-in-water emulsions of the present invention in an aqueous solvent to obtain an aqueous dispersion and an emulsification step including adding the oil phase component to the aqueous dispersion while stirring the aqueous dispersion; and also relates to an oil-in-water emulsion containing the solid emulsifier for oil-in-water emulsions of the present invention.

### Advantageous Effects of Invention

The present invention provides a solid emulsifier for oil-in-water emulsions for use in production of an oil-in-water emulsion that has a less squeaky feeling and a good texture when used as a cosmetic, a method for producing an oil-in-water emulsion, an oil-in-water emulsion, and a cosmetic composition.

### DESCRIPTION OF EMBODIMENTS

The present invention is described in detail below.

The solid emulsifier for oil-in-water emulsions of the present invention contains metal oxide particles having a volume average particle size of 0.2 to 0.4 μm, a specific surface area of 5 to 20 m²/g, and a hydrophobicity of 5 to 35.

Examples of the metal oxide particles include particles of metal oxides such as silica (SiO₂), alumina (Al₂O₃), titania (TiO₂), zinc oxide (ZnO₂), and zirconia (ZrO₂) and particles of complex oxides. Silica particles are preferred.

Herein, semimetals such as silicon (Si) are considered to be included in metals.

When the metal oxide particles have a volume average particle size of 0.2 to 0.4 μm, the emulsion stability increases, and the solid emulsifier for oil-in-water emulsions of the present invention, when used as a cosmetic, does not produce much of a squeaky feeling.

When the metal oxide particles have a specific surface area of 5 to 20 m²/g, the solid emulsifier for oil-in-water emulsions of the present invention, when used as a cosmetic, does not produce much of a squeaky feeling derived from the metal oxide particles.

The specific surface area of the metal oxide particles refers to the BET specific surface area obtained by the BET method. The BET method is a gas adsorption method in which gas particles such as nitrogen are adsorbed onto solid particles and the adsorption amount is used to determine the specific surface area. The specific surface area is determined by calculating the monomolecular adsorption amount VM using the BET equation from the relationship between the pressure P and the adsorption amount V.

Herein, the specific surface area refers to a value measured by the BET multipoint method described in JIS 8830:2013.

The hydrophobicity of the metal oxide particles is more preferably 10 to 32.5, still more preferably 15 to 30, particularly preferably 20 to 30.

The metal oxide particles having a hydrophobicity within the range can stably adsorb onto the interface of oil droplets in water for a long period of time, and can therefore be used as a solid emulsifier for oil-in-water emulsions.

Herein, the hydrophobicity of the “metal oxide particles” refers to a value measured by the following method.

### <Method of Measuring Hydrophobicity>

(1) 50 mL of ion-exchanged water is placed in a 100 mL beaker.
(2) 0.2 g of the metal oxide particles as a measurement sample is added to the beaker in the step (1), followed by stirring with a PTFE stirring bar with a length of 30 mm and a diameter of 8 mm, wherein the stirring speed is adjusted so that when the stirring state in the beaker is observed from a side of the beaker, the lowest point of a vortex created by the stirring is positioned above the midpoint of the distance from the bottom of the beaker to the liquid surface.

The “liquid surface” refers to the liquid surface of ion-exchanged water containing the metal oxide particles in a stationary state before stirring.
(3) 0.5 mL of methanol is added dropwise using a burette with the stirring continued, the stirring is stopped after 30 seconds from the dropwise addition, and 30 seconds after stopping the stirring, the presence or absence of the metal oxide particles at the liquid surface is visually checked.
(4) The step (3) is repeated until the metal oxide particles can no longer be visually observed at the liquid surface.
(5) A percentage of a volume (mL) of the methanol dropwise added in the steps (3) and (4) relative to a sum of a volume of the ion-exchanged water (50 mL) and the volume of the methanol dropwise added in the steps (3) and (4) in the beaker is calculated to determine the hydrophobicity.

Next, a method for producing metal oxide particles that have the above parameters is described.

First, non-surface-treated metal oxide particles having a volume average particle size of 0.2 to 0.4 μm and a specific surface area of 5 to 20 m²/g (hereinafter, also simply referred to as “non-surface-treated metal oxide particles”) are prepared.

The non-surface-treated metal oxide particles can be obtained by a deflagration method in which metal powder is dispersed in an oxygen stream and ignited for oxidation, or by a melting method in which metal powder is melted and spheroidized in a flame. Metal oxide powder obtained by a deflagration method is commonly in the form of spherical particles. Of these methods, the deflagration method is the most preferred, since the surface of the spherical particles obtained can be uniformly chemically modified.

Chemical modification of the surface of such non-surface-treated metal oxide particles with hydrophobic functional groups (hydrocarbon groups, etc.) allows the metal oxide particles to have a hydrophobicity of 5 to 35.

These methods can produce metal oxide particles having the above parameters.

Next, a method for producing metal oxide particles having the above parameters is specifically described with reference to an example of production of silica particles.

First, non-surface-treated silica particles having a volume average particle size of 0.2 to 0.4 μm and a specific surface area of 5 to 20 m²/g are prepared.

Preferred examples of the silica particles include silica particles obtained by a deflagration method and silica particles obtained by a melting method (hereinafter also referred to as “deflagration method silica particles” and “melting method silica particles”, respectively). The silica particles are preferably deflagration method silica particles.

Next, at least part of the surface of the silica particles is chemically modified with hydrophobic functional groups (hydrocarbon groups, etc.), so that the silica particles have a hydrophobicity of 5 to 35.

Such silica particles chemically modified with hydrophobic functional groups (hydrocarbon groups, etc.) can be obtained by surface treatment of silica particles such as deflagration method silica particles or melting method silica particles by silylation of the silanol groups present on the surface of the particles using a known silane coupling agent (alkoxysilane, organosilazane, etc.).

The surface treatment can be performed on dried silica particles or on silica particles in a liquid. In the surface treatment, a surface treatment agent may be added in a vaporized state and reacted, or may be added in a dissolved state in a solvent and reacted (when the surface treatment agent is a liquid, it may be used as is). The solvent and liquid may be a common solvent such as methyl ethyl ketone, acetone, methanol, ethanol, propanol, butanol, or water.

To such silica particles, carbon is preferably attached in an amount of 20 to 80% by mass, more preferably 50 to 80% by mass of the maximum amount of carbon that can be attached to the silica particles for chemical modification. The carbon content can be measured using a carbon analyzer (for example, EMIA-Pro available from HORIBA, Ltd.).

The surface of the silica particles is preferably chemically modified with an alkoxysilane or an organosilazane. Examples of the alkoxysilane include methyltrimethoxysilane, dimethyltrimethoxysilane, phenyltrimethoxysilane, dimethoxydiphenylsilane, n-propyltrimethoxysilane, hexyltrimethoxysilane, tetraethoxysilane, methyltriethoxysilane, dimethyldiethoxysilane, phenyltriethoxysilane, n-propylethoxysilane, hexyltriethoxysilane, and octyltriethoxysilane. Examples of the organosilazane include tetramethyldisilazane, hexamethyldisilazane, and pentamethyldisilazane. From the viewpoint of use in cosmetics, hexamethyldisilazane is most preferred.

The bonds obtained by the reaction between the silanol groups and an organosilazane are resistant to hydrolysis and resistant to breaking over time, so that a solid emulsifier for oil-in-water emulsions that maintains excellent stability over time can be obtained.

The non-surface-treated silica particles may be commercially available ones. For example, the deflagration method silica particles or melting method silica particles may be ADMAFINE SO-C1 (the deflagration method silica particles available from Admatechs Co., Ltd.).

### <Method for Producing Oil-In-Water Emulsion>

A method for producing an oil-in-water emulsion according to a first aspect of the present invention is a method for producing an oil-in-water emulsion containing an aqueous phase component and an oil phase component dispersed in the aqueous phase component, the method including: a dispersion step including dispersing the solid emulsifier for oil-in-water emulsions of the present invention in an aqueous solvent to obtain an aqueous dispersion; and an emulsification step including adding the oil phase component to the aqueous dispersion while stirring the aqueous dispersion.

### Dispersion Step

In this step, the solid emulsifier for oil-in-water emulsions of the present invention is dispersed in an aqueous solvent.

In the dispersion step, an alcohol or a pH adjuster may be added to the aqueous solvent, if necessary.

In this step, the solid emulsifier for oil-in-water emulsions can be dispersed in an aqueous solvent by adding the solid emulsifier for oil-in-water emulsions to the aqueous solvent and stirring them using a known dispersing machine such as a homomixer.

The alcohol or the pH adjuster is preferably added to the aqueous solvent before adding the solid emulsifier for oil-in-water emulsions. In other words, preferably, the alcohol or the pH adjuster is added to the aqueous solvent, and then the solid emulsifier for oil-in-water emulsions is added and dispersed while stirring the aqueous solvent.

The aqueous solvent is preferably an aqueous solution containing water and/or an alcohol.

The water is preferably purified water or ionized water.

The alcohol is preferably one having a water solubility of 500 g/L or higher at 20° C. Examples of the preferred alcohol that can be used include ethanol (infinitely soluble in water), propylene glycol (infinitely soluble in water), dipropylene glycol (infinitely soluble in water), 1,3-butylene glycol (infinitely soluble in water), isopentyldiol (infinitely soluble in water), glycerol (infinitely soluble in water), and polyethylene glycol (PEG-8, etc.) (water solubility at 20° C.: 1000 g/L).

The amount of the alcohol in the aqueous dispersion is preferably 10 to 55 parts by mass relative to 100 parts by mass of water.

The aqueous dispersion obtained by dispersing the solid emulsifier for oil-in-water emulsions preferably has a pH of 7 to 14 at 25° C. The aqueous dispersion having a pH of 7 to 14 at 25° C. is preferred, since the dispersion stability of the solid emulsifier for oil-in-water emulsions increases in the aqueous solvent.

To adjust the pH of the aqueous dispersion to 7 to 14 at 25° C., a pH adjuster may be used. Examples of the pH adjuster that can be used include sodium hydroxide, potassium hydroxide, arginine, triethanolamine, citric acid, ascorbic acid, glycolic acid, and succinic acid.

The mass percentage of the solid emulsifier for oil-in-water emulsions in the aqueous dispersion is preferably 1 to 30% by mass based on the total mass of the aqueous dispersion.

More preferably, the aqueous dispersion contains an alcohol having a water solubility of 500 g/L or higher at 20° C. and has a pH of 7 to 14 at 25° C.

### Emulsification Step

In this step, an oil phase component is added to the aqueous dispersion obtained in the dispersion step while stirring the aqueous dispersion.

The emulsification step may be carried out by gradually adding an oil phase component while stirring the aqueous dispersion with a homomixer at room temperature (preferably 15° C. to 25° C.).

This step can produce an oil-in-water emulsion.

The method for producing an oil-in-water emulsion of the present invention preferably includes, after the emulsification step, an additional aqueous solvent addition step of additionally adding an aqueous solvent under stirring.

The additional aqueous solvent addition step allows the dispersibility of the solid emulsifier for oil-in-water emulsions in water to reduce and allows the solid emulsifier for oil-in-water emulsions to orient towards the oil phase interface.

The method for producing an oil-in-water emulsion of the present invention may include, after the additional aqueous solvent addition step, a water-based thickener addition step of adding a water-based thickener and a pH adjustment step including adding a pH adjuster.

The method may also include a standing step including adjusting the pH of the oil-in-water emulsion to 6.0 to 8.0, defoaming the emulsion, and allowing the emulsion to stand overnight at 25° C.

In the dispersion step in the method for producing an oil-in-water emulsion of the present invention, to the aqueous solvent may be added a water-soluble component that can be added to cosmetics, such as water-soluble UV absorbers, preservatives, moisturizing agents, polymers, amino acids and amino acid derivatives, sugars and sugar derivatives, fragrances, colorants, sequestering agents, antioxidants, and chemical agents.

In the method for producing an oil-in-water emulsion of the present invention, to the oil phase component used in the emulsification step may be added other components that can be added to cosmetics, such as waxes, solid fats, solid waxes, higher fatty acids, oil-soluble UV absorbers, powders, emollients, polymers, amino acid derivatives, sugar derivatives, fragrances, colorants, antioxidants, and biologically active substances.

Next, a method for producing an oil-in-water emulsion according to a second aspect of the present invention is described.

Another aspect of the method for producing an oil-in-water emulsion of the present invention is a method for producing an oil-in-water emulsion, the method including: a dispersion step including dispersing the solid emulsifier for oil-in-water emulsions of the present invention in an oily component to obtain an oil dispersion; and an emulsification step including adding the aqueous phase component to the oil dispersion while stirring the oil dispersion.

In the method for producing an oil-in-water emulsion according to the first aspect of the present invention, the solid emulsifier for oil-in-water emulsions of the present invention is dispersed in an aqueous solvent to obtain an aqueous dispersion, and then an oil phase component is added to the aqueous dispersion. The method for producing an oil-in-water emulsion according to the second aspect of the present invention is different from the previous method for producing an oil-in-water emulsion in that the solid emulsifier for oil-in-water emulsions is dispersed in an oily component to obtain an oil dispersion, and then an aqueous phase component is added.

In this way, even when the solid emulsifier for oil-in-water emulsions of the present invention is first dispersed in an oil phase component, an oil-in-water emulsion can be produced.

The materials and the like used in the method for producing an oil-in-water emulsion according to the second aspect of the present invention are preferably the same as the materials and the like used in the method for producing an oil-in-water emulsion according to the first aspect of the present invention.

The method for producing an oil-in-water emulsion of the present invention can produce an oil-in-water emulsion containing the solid emulsifier for oil-in-water emulsions of the present invention.

The present invention encompasses an oil-in-water emulsion containing the solid emulsifier for oil-in-water emulsions of the present invention.

### <Oil-In-Water Emulsion>

The oil-in-water emulsion of the present invention contains the solid emulsifier for oil-in-water emulsions of the present invention.

The oil-in-water emulsion contains an aqueous phase component and an oil phase component dispersed in the aqueous phase component.

The aqueous phase component is preferably contained in an amount of 30 to 99% by volume of the total amount of the oil-in-water emulsion, and the oil phase component is preferably contained in an amount of 1 to 70% by volume of the total amount of the oil-in-water emulsion. The aqueous phase component includes an aqueous solvent, and may further include a water-based thickener and a pH adjuster if necessary.

Examples of the aqueous solvent include purified water, ionized water, and purified water or ionized water containing alcohol. Examples of the alcohol include monohydric alcohols such as ethanol, 3-methoxy-1-butanol, and 3-methoxy-3-methyl-1-butanol; dihydric alcohols such as propylene glycol, dipropylene glycol, 1,3-butylene glycol, isopentyldiol, and tetraethylene glycol; polyhydric alcohols such as glycerol, diglycerol, polyglycerol, and polyethylene glycol (PEG-8, etc.); and sugars and sugar derivatives such as trehalose and glucosyltrehalose.

From the viewpoint of the dispersibility of the solid emulsifier, preferred are ethanol, propylene glycol, dipropylene glycol, 1,3-butylene glycol, isopentyldiol, glycerol, and polyethylene glycol.

Examples of the water-based thickener include water-soluble natural polymers, semi-synthetic polymers, synthetic polymers, and clay minerals. Examples of the natural polymers include xanthan gum, guar gum, and cellulose nanofibers. Examples of the semi-synthetic polymers include polysaccharide derivatives such as methyl cellulose, ethyl cellulose, hydroxyethyl cellulose, and carboxymethyl cellulose. Examples of the synthetic polymers include polyvinyl alcohol, polyvinylpyrrolidone, polyacrylic acid, and sodium acrylate grafted starch. Examples of the clay minerals include bentonite and smectite. Of these, hydroxyethyl cellulose, cellulose nanofibers, polyacrylic acid, and sodium acrylate grafted starch are preferred. The amount of the thickener is preferably about 0.01% by mass to 5% by mass relative to the total amount of the oil-in-water emulsion.

The pH adjuster may be, for example, sodium hydroxide, potassium hydroxide, arginine, triethanolamine, citric acid, ascorbic acid, glycolic acid, or succinic acid. The amount of the pH adjuster is preferably 1% by mass or less relative to the total amount of the oil-in-water emulsion.

The aqueous phase component in the oil-in-water emulsion of the present invention may contain a water-soluble component that can be added to cosmetics, such as water-soluble UV absorbers, preservatives, moisturizing agents, polymers, amino acids and amino acid derivatives, sugars and sugar derivatives, fragrances, colorants, sequestering agents, antioxidants, and chemical agents, as long as they do not impair the effects of the present invention.

The oil phase component may be an oily component that can be added to cosmetics, and is preferably at least one selected from the group consisting of natural fats and oils, silicone oils, hydrocarbon oils, and synthetic ester oils.

Examples of the natural fats and oils include avocado oil, camellia oil, turtle oil, macadamia nut oil, corn oil, mink oil, olive oil, rapeseed oil, egg yolk oil, sesame oil, persic oil, wheat germ oil, camellia sasanqua oil, castor oil, linseed oil, safflower oil, cottonseed oil, perilla oil, soybean oil, peanut oil, tea seed oil, Torreya nucifera seed oil, rice bran oil, Chinese wood oil, Japanese wood oil, jojoba oil, and germ oil.

Examples of the silicone oils include cyclic polysiloxanes such as cyclopentasiloxane, octamethylcyclotetrasiloxane, decamethylcyclopentasiloxane, and dodecamethylcyclohexasiloxane; chain polysiloxanes such as dimethylpolysiloxane, methylphenylpolysiloxane, diphenylpolysiloxane, and caprylyl methicone; modified polysiloxanes such as amino-modified polysiloxane, polyether-modified polysiloxane, polyether-modified dimethylpolysiloxane, alkyl-modified polysiloxane, and fluorine-modified polysiloxane; and crosslinked products thereof.

Examples of the hydrocarbon oils include isododecane, isohexadecane, squalane, squalene, paraffin, light isoparaffin, liquid paraffin, hydrogenated poly(C6-12 olefin), and pristane.

Examples of the synthetic ester oils include triethylhexanoin, isopropyl myristate, octyldodecyl myristate, isopropyl palmitate, hexyl laurate, decyl oleate, hexyldecyl dimethyloctanoate, lanolin acetate, isocetyl stearate, hexyldecyl isostearate, cetyl ethylhexanoate, neopentyl glycol dicaprate, diisostearyl malate, glyceryl diisostearate, trimethylolpropane triethylhexanoate, trimethylolpropane triisostearate, pentaerythrityl tetraethylhexanoate, triethylhexanoin, triisopalmitin, trimethylolpropane triisostearate, ethylhexyl palmitate, trimyristin, oleyl oleate, diethylhexyl sebacate, isocetyl myristate, hexyldecyl palmitate, diisopropyl sebacate, diethylhexyl succinate, ethylhexyl palmitate, and caprylic/capric triglyceride.

The oil phase component in the oil-in-water emulsion of the present invention may contain other components that can be added to cosmetics, such as waxes, solid fats, solid waxes, higher fatty acids, oil-soluble UV absorbers, powders, emollients, polymers, amino acid derivatives, sugar derivatives, fragrances, colorants, antioxidants, and biologically active substances, as long as they do not impair the effects of the present invention.

### <Cosmetic Composition>

The cosmetic composition of the present invention contains the oil-in-water emulsion of the present invention.

As long as the effects of the present invention are not impaired, the cosmetic composition of the present invention may appropriately contain known components that can be used in cosmetics, depending on the application and purpose.

Examples of the known components that can be incorporated into the cosmetic composition of the present invention include oil components, surfactants, water-miscible organic solvents, thickeners, inorganic powders, acids, alkalis, and sugars.

Examples of the oil components include oil waxes, hydrocarbons, higher fatty acids, higher alcohols, and esters.

Examples of the oil waxes include almond oil, olive oil, hardened oil, camellia oil, castor oil, coconut oil, silicone oil, beeswax, lanolin, and carnauba wax.

The hydrocarbons include linear or branched hydrocarbon oils, and specific examples thereof include squalane, ceresin, paraffin, liquid paraffin, and petrolatum.

The higher fatty acids include C12-C30 fatty acids, and specific examples thereof include lauric acid, myristic acid, palmitic acid, stearic acid, behenic acid, undecylenic acid, oleic acid, linoleic acid, linolenic acid, arachidonic acid, eicosapentaenoic acid (EPA), docosahexaenoic acid (DHA), isostearic acid, and 12-hydroxystearic acid.

The higher alcohols include C8-C30 alcohols, and specific examples thereof include lauryl alcohol, myristyl alcohol, palmityl alcohol, stearyl alcohol, behenyl alcohol, hexadecyl alcohol, oleyl alcohol, isostearyl alcohol, hexyldodecanol, octyldodecanol, cetostearyl alcohol, 2-decyltetradecynol, cholesterol, phytosterol, polyoxyethylene cholesterol ether, monostearyl glyceryl ether (batyl alcohol), and monooleyl glyceryl ether (selachyl alcohol).

The esters include C3-C100 ester oils, and specific examples thereof include butyl stearate, cetyl lactate, myristyl lactate, isopropyl palmitate, isopropyl myristate, octyldodecyl myristate, myristyl myristate, hexyl laurate, glyceryl monostearate, and glyceryl di-2-heptylundecanoate.

The surfactants include nonionic surfactants, anionic surfactants, cationic surfactants, and amphoteric surfactants.

Examples of the nonionic surfactants include aliphatic alcohol (number of carbon atoms: 8 to 24) alkylene oxide (number of carbon atoms: 2 to 8) adducts (degree of polymerization=1 to 100) [an adduct of 11 mol of ethylene oxide with oleyl alcohol, etc.], (poly)oxyalkylene (number of carbon atoms: 2 to 8, degree of polymerization=1 to 100) glycol higher fatty acid (number of carbon atoms: 8 to 24) esters [polyethylene glycol monostearate (degree of polymerization=20), polyethylene glycol distearate (degree of polymerization=30), etc.], polyhydric (2 to 10 or more hydroxy groups) alcohol fatty acid (number of carbon atoms: 8 to 24) esters [propylene glycol monolaurate, glycerol monostearate, ethylene glycol monostearate, sorbitan monolaurate, etc.], polyhydric (2 to 10 or more hydroxy groups) alcohol higher fatty acid (number of carbon atoms: 8 to 24) ester (poly)alkylene oxide adducts (the alkylene group has 2 to 8 carbon atoms, degree of polymerization=1 to 100) [an adduct of ethylene oxide with sorbitan monolaurate (degree of polymerization=10), an adduct of ethylene oxide with methyl glucose dioleate (degree of polymerization=50), etc.], fatty acid N-hydroxyalkylamides [1:1 type coconut oil fatty acid diethanolamide, 1:1 type lauric acid diethanolamide, etc.], alkyl (number of carbon atoms: 1 to 22) (poly)oxyalkylene (number of carbon atoms: 2 to 8, degree of polymerization=1 to 100) phenyl ethers, alkyl (number of carbon atoms: 8 to 24) (poly)oxyalkylene (number of carbon atoms: 2 to 8, degree of polymerization=1 to 100)-aminoalkyl (number of carbon atoms: 8 to 24)-ethers, and alkyl (number of carbon atoms: 8 to 24) dialkyl (number of carbon atoms: 1 to 6) amine oxides [lauryl dimethylamine oxide, etc.].

Examples of the anionic surfactants include C8-C24 alkyl ether carboxylic acids or salts thereof, C8-C24 alkyl (poly)oxyethylene ether carboxylic acids or salts thereof [sodium (poly)oxyethylene (degree of polymerization=1 to 100) lauryl ether acetate, disodium (poly)oxyethylene (degree of polymerization=1 to 100) lauryl sulfosuccinate, etc.], C8-C24 alkyl sulfates and C8-C24 alkyl (poly)oxyethylene sulfates [sodium lauryl sulfate, sodium lauryl (poly)oxyethylene (degree of polymerization=1 to 100) sulfate, lauryl (poly)oxyethylene (degree of polymerization=1 to 100) sulfate-triethanolamine salts, etc.], sodium coconut oil fatty acid monoethanolamide sulfate sulfonate, C8-C24 alkylphenyl sulfonates [sodium dodecylbenzenesulfonate, etc.], C8-C24 alkyl phosphates and C8-C24 alkyl (poly)oxyethylene phosphates [sodium lauryl phosphate, sodium (poly)oxyethylene (degree of polymerization=1 to 100) lauryl ether phosphate, etc.], fatty acid salts [sodium laurate, triethanolamine laurate, etc.], acylated amino acid salts [sodium coconut oil fatty acid methyl taurate, sodium coconut oil fatty acid sarcosine, triethanolamine coconut oil fatty acid sarcosine, triethanolamine N-coconut oil fatty acid acyl-L-glutamate, sodium N-coconut oil fatty acid acyl-L-glutamate, sodium lauroyl methyl-β-alanine, etc.].

Examples of the cationic surfactants include quaternary ammonium salt type surfactants [stearyl trimethyl ammonium chloride, behenyl trimethyl ammonium chloride, distearyl dimethyl ammonium chloride, lanolin fatty acid aminopropyl ethyl dimethyl ammonium ethyl sulfate, etc.] and amine salt type surfactants [stearic acid diethylaminoethylamide lactate, dilaurylamine hydrochloride, oleylamine lactate, etc.].

Examples of the amphoteric surfactants include betaine-type amphoteric surfactants [coconut oil fatty acid amidopropyl dimethylaminoacetate betaine, lauryl dimethylaminoacetate betaine, 2-alkyl-N-carboxymethyl-N-hydroxyethyl imidazolinium betaine, lauryl hydroxysulfobetaine, sodium lauroyl amidoethyl hydroxyethyl carboxymethyl betaine hydroxypropyl phosphate, etc.] and amino acid-type amphoteric surfactants [sodium β-lauryl aminopropionate, etc.].

Specific examples of the water-miscible organic solvents include ethanol, isopropyl alcohol, ethylene glycol, diethylene glycol, triethylene glycol, polyethylene glycol, propylene glycol, dipropylene glycol, polypropylene glycol, glycerol, diglycerol, polyglycerol, 3-methyl-1,3-butanediol, 1,3-butanediol, 1,2-pentanediol, and 1,2-hexanediol.

Examples of the thickeners include guar gum, starch, carboxymethylcellulose, acetylmethylcellulose, polyvinyl alcohol, and sodium polyacrylate.

Examples of the inorganic powders include zinc oxide, titanium oxide, barium sulfate, calcium sulfate, calcium carbonate, talc, mica, kaolin, silicic acid, aluminum silicate, hydroxyapatite, vermiculite, higilite, bentonite, montmorillonite, hectorite, zeolite, ceramic powder, alumina, silica, and silica silylate.

Examples of the acids include citric acid, tartaric acid, lactic acid, and phosphoric acid. Examples of the alkalis include hydroxides of alkali metals (sodium, potassium, etc.) or alkaline earth metals (calcium, etc.), aqueous ammonia, triethanolamine, diethanolamine, and monoethanolamine.

Examples of the sugars include mannitol, sorbitol, xylitol, maltitol, erythritol, pentaerythritol, glucose, sucrose, fructose, lactose, maltose, xylose, and trehalose.

In addition to the above components, the cosmetic herein may contain components used in common cosmetics [components described in “Saishin Keshohin Kagaku” [Latest Cosmetic Science](Yakuji Nippo, Limited) such as colorants, fragrances, hormones, vitamins, photosensitizers, plant and animal extracts, UV absorbers, antioxidants, preservatives/antimicrobial agents, and antiperspirants/deodorants].

The cosmetic composition of the present invention is suitable for use in skin care, hair care, and makeup applications because it can provide a good texture when used. The cosmetic composition of the present invention may be in the form of a spray, lotion, sheet, powder, or the like.

Specific examples of the applications include facial cleansers, cleaning agents, makeup removers, toners, moisturizers, gels, serums, face packs, face masks, shaving lotions, sunscreens, after-sun lotions, tanning products, deodorant lotions, body lotions, hand care lotions, foot care lotions, lip balms, lip glosses, lip liners, hair waxes, hair mousses, hair gels, hair oils, hair sprays, hair mists, hair lotions, leave-in treatments, leave-in conditioners, powder foundations, liquid foundations, concealers, blushes, eyeliners, eyebrow products, eyeshadows, and lipsticks.

The following contents are disclosed herein.

Disclosure <1> relates to a solid emulsifier for oil-in-water emulsions, the solid emulsifier containing metal oxide particles having a volume average particle size of 0.2 to 0.4 μm, a specific surface area of 5 to 20 m²/g, and a hydrophobicity of 5 to 35, with the hydrophobicity being measured by the following method:

### <Method of Measuring Hydrophobicity>

(1) 50 mL of ion-exchanged water is placed in a 100 mL beaker;
(2) 0.2 g of the metal oxide particles as a measurement sample is added to the beaker in the step (1), followed by stirring with a PTFE stirring bar with a length of 30 mm and a diameter of 8 mm, wherein a stirring speed is adjusted so that when a stirring state in the beaker is observed from a side of the beaker, a lowest point of a vortex created by the stirring is positioned above the midpoint of a distance from a bottom of the beaker to a liquid surface;
(3) 0.5 mL of methanol is added dropwise using a burette with the stirring continued, the stirring is stopped after 30 seconds from the dropwise addition, and 30 seconds after stopping the stirring, the presence or absence of the metal oxide particles at the liquid surface is visually checked;
(4) the step (3) is repeated until the metal oxide particles can no longer be visually observed at the liquid surface; and
(5) a percentage of a volume (mL) of the methanol dropwise added in the steps (3) and (4) relative to a sum of a volume (50 mL) of the ion-exchanged water and the volume of the methanol dropwise added in the steps (3) and (4) in the beaker is calculated to determine the hydrophobicity.

Disclosure <2> relates to the solid emulsifier for oil-in-water emulsions according to Disclosure <1>,
wherein the metal oxide particles are silica particles.

Disclosure <3> relates to a method for producing an oil-in-water emulsion containing an aqueous phase component and an oil phase component dispersed in the aqueous phase component, the method including:
a dispersion step including dispersing the solid emulsifier for oil-in-water emulsions according to Disclosure <1> or <2> in an aqueous solvent to obtain an aqueous dispersion; and
an emulsification step including adding the oil phase component to the aqueous dispersion while stirring the aqueous dispersion.

Disclosure <4> relates to the method for producing an oil-in-water emulsion according to Disclosure <3>,
wherein the aqueous dispersion contains an alcohol having a water solubility of 500 g/L or higher at 20° C., a mass ratio of the alcohol in the aqueous dispersion is 5 to 55 parts by mass relative to 100 parts by mass of water, and the aqueous dispersion has a pH of 7 to 14 at 25° C.

Disclosure <5> relates to a method for producing an oil-in-water emulsion containing an aqueous phase component and an oil phase component dispersed in the aqueous phase component, the method including:
a dispersion step including dispersing the solid emulsifier for oil-in-water emulsions according to Disclosure <1> or <2> in an oily component to obtain an oil dispersion; and
an emulsification step including adding the aqueous phase component to the oil dispersion while stirring the oil dispersion.

Disclosure <6> relates to an oil-in-water emulsion containing the solid emulsifier for oil-in-water emulsions according to Disclosure <1> or <2>.

Disclosure <7> relates to a cosmetic composition containing the oil-in-water emulsion according to Disclosure <6>.

### EXAMPLES

The present invention is described in detail below with reference to examples, but the present invention is not limited to these examples. The following describes the methods of measuring the parameters.

### <Volume Average Particle Size>

In the examples and comparative examples described later, silica particles were used as non-surface-treated metal oxide particles. The volume average particle size of the silica particles was measured using a meter such as a laser diffraction particle size distribution meter (HORIBA, Ltd.). A measuring cell of the laser diffraction particle size distribution meter was filled with a solvent such as water or isopropyl alcohol, and silica powder was added to the cell little by little while applying ultrasonic waves until the concentration reached a level appropriate for measurement. After adjusting the concentration to an appropriate level, the particle size distribution was measured.

<BET Specific Surface Area (m²/g)>

The BET specific surface area of the silica particles was measured by the BET method described in JIS 8830:2013 using a gas adsorption specific surface area analyzer (Shimadzu Corporation). The silica particles were weighed in a measuring cell of the surface area analyzer, heated to 200° C., cooled with liquid nitrogen, and heated. The specific surface area was calculated from the amount of nitrogen gas desorbed.

### <Production of Solid Emulsifier for Oil-In-Water Emulsion>

### Example 1-1

Deflagration method silica particles without surface treatment (trade name: “SO-C1”, volume average particle size: 0.3 μm, specific surface area: 16 m²/g, manufacturer: Admatechs Co., Ltd.) in an amount of 100 parts by mass were surface-treated with 0.75 parts by mass of a silylating agent (Shin-Etsu Chemical Co., Ltd., trade name: “SZ-31” hexamethyldisilazane) to obtain a solid emulsifier (E-1) for oil-in-water emulsions of Example 1-1. The solid emulsifier had a hydrophobicity of 35.

The surface treatment with the silylating agent was carried out by adding the silylating agent little by little while stirring the silica powder with a mixer, and then heating the mixture at 130° C.

### (Example 1-2), (Example 1-3), (Comparative Example 1-1), and (Comparative Example 1-2)

Solid emulsifiers (E-2 and E-3) for oil-in-water emulsions according to Examples 2 and 3 and solid emulsifiers (CE-1 and CE-2) for oil-in-water emulsions according to Comparative Examples 1 and 2 were prepared as in Example 1-1, except that the amounts of the silica particles and the silylating agent were changed to those shown in Table 1. The hydrophobicity of the resulting solid emulsifiers for oil-in-water emulsion was measured. Table 1 shows the results. The unit of the numerical values of the components in Table 1 is “parts by mass”.

TABLE 1

| | | Example | | | Comparative Example | |
|---|---|---|---|---|---|---|
| | | 1-1 | 1-2 | 1-3 | 1-1 | 1-2 |
| Solid emulsifier for oil-in-water emulsion | | E-1 | E-2 | E-3 | CE-1 | CE-2 |
| Silica particle | | 100 | 100 | 100 | 100 | 100 |
| Silylating agent | Hexamethyldisilazane | 0.750 | 0.627 | 0.380 | 0.812 | 0.349 |
| Evaluation | Hydrophobicity | 35 | 25 | 5 | 40 | 2.5 |

### <Production of Oil-In-Water Emulsion>

### Example 2-1

### 1. Dispersion Step

A 300 mL tall beaker was charged with 35.4 g of ionized water and 6.25 g of ethanol, and a 10% by mass aqueous sodium hydroxide solution (0.1417 g) as an aqueous solution of a pH adjuster was added thereto. The contents were homogenized. Thereafter, 3.75 g of the solid emulsifier (E-1) for oil-in-water emulsions obtained in Example 1-1 was added, followed by stirring for one minute (rotation speed: 8000 rpm) using a homomixer to produce an aqueous dispersion.

### 2. Emulsification Step

After producing the aqueous dispersion, 25 g of liquid paraffin (Sonneborn LLC, trade name: “Carnation”) was added as an oily component while maintaining stirring. One minute later, 54.2 g of ionized water serving as another aqueous solvent. One minute after that, 125 g of 1% by mass hydroxyethyl cellulose (Tokyo Chemical Industry Co., Ltd., trade name: “Hydroxyethyl Cellulose” (4500 to 6500 mPa·s, 2% by mass in water at 25° C.)) was added as an aqueous solution of a water-based thickener. While maintaining the stirring, 0.2268 g of a 10% by mass aqueous citric acid solution (Tokyo Chemical Industry Co., Ltd.) serving as a pH adjuster for the different water-soluble component was further added thereto, so that the pH was 7.0±1.0.

### 3. Defoaming and Allowing to Stand

After the emulsification step, the mixture was defoamed and then allowed to stand overnight at 25° C. to produce an oil-in-water emulsion of Example 2-1.

### (Example 2-2) to (Example 2-8) and (Comparative Example 2-1) to (Comparative Example 2-3)

Oil-in-water emulsions of Examples 2-2 to 2-8 and Comparative Examples 2-1 to 2-3 were prepared as in Example 2-1, except that the amounts and types of the solid emulsifier for oil-in-water emulsions, the aqueous solvents, the aqueous solution of a pH adjuster, the different water-soluble components, the aqueous solution of a thickener, and the oily component were changed to those shown in Tables 2 and 4. Comparative Example 2-3 used commercially available surface-treated fumed silica particles (Evonik, trade name: “Aerosil r816”, volume average particle size: 0.01 μm, specific surface area: 170 to 210 m²/g, hydrophobicity: 0) as a solid emulsifier (CE-3) for oil-in-water emulsions.

### Example 2-9

### 1. Emulsification Step

A 300 mL tall beaker was charged with 50 g of liquid paraffin (Sonneborn LLC, trade name: “Carnation”) as an oily component and 7.5 g of the solid emulsifier (E-2) for oil-in-water emulsions obtained in Example 1-2. The contents were stirred with a homomixer (rotation speed: 8000 rpm) for one minute, and 67.5 g of ionized water was added thereto. One minute later, 125 g of 1% by mass hydroxyethyl cellulose (Tokyo Chemical Industry Co., Ltd., trade name: “Hydroxyethyl Cellulose” (4500 to 6500 mPa·s, 2% by mass in water at 25° C.)) was added as an aqueous solution of a water-based thickener, followed by stirring for one minute.

### 2. Defoaming and Allowing to Stand

After the emulsification step, the mixture was defoamed and then allowed to stand overnight at 25° C. to produce an oil-in-water emulsion of Example 2-9.

The oil-in-water emulsions of the examples and comparative examples were subjected to an appearance test, a storage stability test, and an evaluation of texture when used. Tables 2 to 4 show the results.

The unit of the numerical values of the components in Tables 2 to 4 is “g”.

### <Appearance Test>

A 40-mL portion of the obtained oil-in-water emulsion was placed in a 50 ml screw-cap tube, and the presence or absence of an aqueous phase and an oil phase was checked. Evaluation was performed using the following criteria.

◯ (Good): Only the O/W emulsion phase was observed, with no separation observed.

x (Poor): In addition to the emulsion phase, the oil phase was observed.

### <Storage Stability Test>

A 40-mL portion of the obtained oil-in-water emulsion was placed in a 50 mL screw-cap tube and stored at 25° C. or 50° C. for one month. Thereafter, the separation of the oil phase was checked. Evaluation was performed using the following criteria.

### (Evaluation Criteria)

◯ (Good): No separation of the oil phase occurred.
x (Poor): Separation of the oil phase occurred.
<Evaluation of Texture when Used>

The evaluation of the squeaky feeling on the skin was conducted by 10 expert panelists. Each panelist washed their inner forearm with water, wiped off water with a towel, and dried for 15 minutes. Then, 1 g of any of the obtained oil-in-water emulsions was applied to the inner forearm. The squeaky feeling on the skin was evaluated through sensory testing, the results were expressed as a rating, and the average rating was used for the evaluation. The squeaky feeling of the cosmetics during use was evaluated through sensory testing on a rating from 1 to 5, and the average rating was calculated from the 10 panelists. Evaluation was performed using the following criteria.
5: No squeaky feeling and very good texture when used
4: No squeaky feeling and good texture when used
3: Acceptable squeaky feeling and texture
2: A slight squeaky feeling
1: A strong squeaky feeling and unpleasant texture when used

TABLE 2

| | | | | Example | | | |
|---|---|---|---|---|---|---|---|
| | | | | 2-1 | 2-2 | 2-3 | 2-4 |
| Aqueous | Aqueous | Solid emulsifier | E-1 | 3.75 | — | — | — |
| phase | dispersion | for oil-in-water | E-2 | — | — | 10 | 7.5 |
| component | | emulsion | E-3 | — | 10 | — | — |
| | | | CE-1 | — | — | — | — |
| | | | CE-2 | — | — | — | — |
| | | | CE-3 | — | — | — | — |
| | | Aqueous solvent | Ionized water | 35.4 | 35.4 | 70.8 | 23.2 |
| | | | Ethanol | 6.25 | — | — | — |
| | | | Propylene glycol | — | 6.25 | — | — |
| | | | Dipropylene glycol | — | — | 12.5 | — |
| | | | 1,3-Butylene glycol | — | — | — | 12.5 |
| | | | Isopentyldiol | — | — | — | — |
| | | | Glycerol | — | — | — | — |
| | | | PEG-8 | — | — | — | — |
| | | pH adjuster | 10% by mass aqueous sodium | 0.1417 | — | — | — |
| | | | hydroxide solution | | | | |
| | | | 10% by mass aqueous potassium | — | 0.0199 | 0.0397 | 0.1302 |
| | | | hydroxide solution | | | | |
| | | pH | | 8.57 | 7.45 | 7.38 | 7.42 |
| | Different | Aqueous solvent | Ionized water | 54.2 | 23.3 | 29.6 | 106.5 |
| | aqueous phase | Water-based | 1% by mass hydroxyethyl cellulose | 125 | — | — | — |
| | component | thickener | 1% by mass cellulose nanofiber | — | 125 | — | — |
| | | | 1% by mass polyacrylic acid | — | — | 50 | — |
| | | | 1% by mass sodium acrylate grafted starch | — | — | — | 50 |
| | | pH adjuster | 10% by mass aqueous potassium | — | — | 2 | — |
| | | | hydroxide solution | | | | |
| | | | 10% by mass aqueous citric acid solution | 0.2268 | 0.0227 | — | 0.1487 |

| | | | | | | |
|---|---|---|---|---|---|---|
| Oil | Oily component | Liquid paraffin | 25 | — | — | — |
| phase | | Dimethicone | — | 50 | — | — |
| component | | Caprylic/capric triglyceride | — | — | 75 | — |

| | Triethylhexanoin | — | — | — | 50 |
|---|---|---|---|---|---|
| | Olive oil | — | — | — | — |
| | Cetyl ethylhexanoate | — | — | — | — |
| Evaluation | pH | 6.53 | 6.81 | 6.34 | 6.70 |
| | Appearance test | ∘ | ∘ | ∘ | ∘ |
| | Storage stability test (25° C., 1 month) | ∘ | ∘ | ∘ | ∘ |
| | Storage stability test (50° C., 1 month) | ∘ | ∘ | ∘ | ∘ |
| | Evaluation of texture when used (squeakiness) | 4.7 | 4.5 | 4.1 | 4.2 |

| | | | | Example | | | |
|---|---|---|---|---|---|---|---|
| | | | | 2-5 | 2-6 | 2-7 | 2-8 |
| Aqueous | Aqueous | Solid emulsifier | E-1 | — | — | — | — |
| phase | dispersion | for oil-in-water | E-2 | 7.5 | 7.5 | 7.5 | 7.5 |
| component | | emulsion | E-3 | — | — | — | — |
| | | | CE-1 | — | — | — | — |
| | | | CE-2 | — | — | — | — |
| | | | CE-3 | — | — | — | — |
| | | Aqueous solvent | Ionized water | 37.5 | 37.5 | 37.5 | 50.0 |
| | | | Ethanol | — | — | — | — |
| | | | Propylene glycol | — | — | — | — |
| | | | Dipropylene glycol | — | — | — | — |
| | | | 1,3-Butylene glycol | — | — | — | — |
| | | | Isopentyldiol | 12.5 | — | — | — |
| | | | Glycerol | — | 12.5 | — | — |
| | | | PEG-8 | — | — | 12.5 | — |
| | | pH adjuster | 10% by mass aqueous sodium | — | — | — | — |
| | | | hydroxide solution | | | | |
| | | | 10% by mass aqueous potassium | 0.0665 | 0.2104 | 0.2104 | — |
| | | | hydroxide solution | | | | |

| | pH | | 8.11 | 9.21 | 9.14 | 6.15 |
|---|---|---|---|---|---|---|
| Different | Aqueous solvent | Ionized water | 17.4 | 17.0 | 17.0 | 17.5 |
| aqueous phase | Water-based | 1% by mass hydroxyethyl cellulose | 125 | 125 | 125 | 125 |
| component | thickener | 1% by mass cellulose nanofiber | — | — | — | — |
| | | 1% by mass polyacrylic acid | — | — | — | — |
| | | 1% by mass sodium acrylate grafted starch | — | — | — | — |
| | pH adjuster | 10% by mass aqueous potassium | — | — | — | — |
| | | hydroxide solution | | | | |
| | | 10% by mass aqueous citric acid solution | 0.0759 | 0.2402 | 0.2402 | — |

| | | | | | | |
|---|---|---|---|---|---|---|
| Oil | Oily component | Liquid paraffin | — | — | 50 | 50 |
| phase | | Dimethicone | — | — | — | — |
| component | | Caprylic/capric triglyceride | — | — | — | — |

| | Triethylhexanoin | — | — | — | — |
|---|---|---|---|---|---|
| | Olive oil | 50 | — | — | — |
| | Cetyl ethylhexanoate | — | 50 | — | — |
| Evaluation | pH | 6.45 | 6.48 | 6.26 | 6.19 |

| | | | | |
|---|---|---|---|---|
| Appearance test | ∘ | ∘ | ∘ | ∘ |
| Storage stability test (25° C., 1 month) | ∘ | ∘ | ∘ | ∘ |
| Storage stability test (50° C., 1 month) | ∘ | ∘ | ∘ | ∘ |
| Evaluation of texture when used (squeakiness) | 4.1 | 4.3 | 4.6 | 3.8 |

TABLE 3

| | | | Example |
|---|---|---|---|
| | | | 2-9 |
| Oil phase | Solid emulsifier | E-2 | 7.5 |
| component | for oil-in-water | | |
| | emulsion | | |
| | Oily component | Liquid paraffin | 50 |
| Aqueous | Aqueous solvent | Ionized water | 67.5 |
| phase | Water-based | 1% by mass hydroxyethyl | 125 |
| component | thickener | cellulose | |

| Evaluation | ph | 6.38 |
|---|---|---|
| | Appearance test | ∘ |
| | Storage stability test | ∘ |
| | (25° C., 1 month) | |
| | Storage stability test | ∘ |
| | (50° C., 1 month) | |
| | Evaluation of texture when | 3.6 |
| | used (squeakiness) | |

TABLE 4

| | | | | Comparative Example | | |
|---|---|---|---|---|---|---|
| | | | | 2-1 | 2-2 | 2-3 |
| Aqueous | Aqueous | Solid emulsifier | E-1 | — | — | — |
| phase | dispersion | for oil-in-water | E-2 | — | — | — |
| component | | emulsion | E-3 | — | — | — |
| | | | CE-1 | 7.5 | — | — |
| | | | CE-2 | — | 7.5 | — |
| | | | CE-3 | — | — | 7.5 |
| | | Aqueous solvent | Ionized water | 37.5 | 37.5 | 37.5 |
| | | | Ethanol | — | — | — |
| | | | Propylene glycol | — | — | — |
| | | | Dipropylene glycol | — | — | — |
| | | | 1,3-Butylene glycol | — | — | — |
| | | | Isopentyldiol | 12.5 | 12.5 | 12.5 |
| | | | Glycerol | — | — | — |
| | | | PEG-8 | — | — | — |
| | | pH adjuster | 10% by mass aqueous sodium hydroxide solution | — | — | — |
| | | | 10% by mass aqueous potassium hydroxide solution | 0.2104 | 0.0210 | 0.0210 |
| | | pH | | 9.24 | 7.43 | 7.32 |
| | Different | Aqueous solvent | Ionized water | 17.0 | 17.5 | 17.5 |
| | aqueous phase | Water-based | 1% by mass hydroxyethyl cellulose | 125 | 125 | 125 |
| | component | thickener | 1% by mass cellulose nanofiber | — | — | — |
| | | | 1% by mass polyacrylic acid | — | — | — |
| | | | 1% by mass sodium acrylate grafted starch | — | — | — |
| | | pH adjuster | 10% by mass aqueous potassium hydroxide solution | — | — | — |
| | | | 10% by mass aqueous citric acid solution | 0.2402 | 0.0240 | 0.0240 |

| | | | | | |
|---|---|---|---|---|---|
| Oil | Oily component | Liquid paraffin | 50 | 50 | 50 |
| phase | | Dimethicone | — | — | — |
| component | | Caprylic/capric triglyceride | — | — | — |

| | Triethylhexanoin | — | — | — |
|---|---|---|---|---|
| | Olive oil | — | — | — |
| | Cetyl ethylhexanoate | — | — | — |
| Evaluation | pH | 6.61 | 6.57 | 6.42 |
| | Appearance test | x | ∘ | ∘ |
| | Storage stability test (25° C., 1 month) | — | x | x |
| | Storage stability test (50° C., 1 month) | — | x | x |
| | Evaluation of texture when used (squeakiness) | — | 4 | 1.6 |

The raw materials shown in Tables 2 to 4 other than those described in the examples are as follows.

### Aqueous Solvent in Aqueous Dispersion

Ethanol (FUJIFILM Wako Pure Chemical Corporation, trade name: “Ethanol (99.5)”)
Propylene glycol (FUJIFILM Wako Pure Chemical Corporation, trade name: “Propylene glycol”)
Dipropylene glycol (Adeka Corporation, trade name: “DPG-RF”)
1,3-Butylene glycol (Tokyo Chemical Industry Co., Ltd., trade name: “1,3-Butanediol”)
Isopentyldiol (Kuraray Co., Ltd., trade name “IPG-S”)
Glycerol (Kao Corporation, trade name: “GLYCERIN (COSMETIC GRADE)”))
PEG-8 (Sanyo Chemical Industries, Ltd., trade name: “PEG-400”)

### pH Adjuster in Aqueous Dispersion

Potassium hydroxide (FUJIFILM Wako Pure Chemical Corporation, trade name: “Potassium hydroxide”) Thickener in different water-soluble component
Cellulose nanofibers (Nippon Paper Industries Co., Ltd., trade name: “Cellenpia CS-01C”)
Polyacrylic acid (Lubrizol, trade name: “Carbopol 980 Polymer”)
Sodium acrylate grafted starch (Sanyo Chemical Industries, Ltd., trade name: “Sunfresh ST-100”) Oily component
Dimethicone (dimethylpolysiloxane) (Shin-Etsu Chemical Co., Ltd., trade name: “KF-96L-2cs”)
Caprylic/capric triglyceride (The Nisshin OilliO Group, Ltd., trade name: “O.D.O”)
Triethylheisanoin (The Nisshin OilliO Group, Ltd., trade name: “T.I.O”)
Olive oil (Nikko Chemicals Co., Ltd., trade name: “NIKKOL Olive Oil”)
Cetyl ethylhexanoate (The Nisshin OilliO Group, Ltd., trade name: “Salacos 816”)

In Tables 2 to 4, the numerical values of the components are expressed in “g”.

As shown in Tables 2 to 4, the oil-in-water emulsions of the examples had higher storage stability, a lower squeaky feeling, and a better texture when used than the oil-in-water emulsions of the comparative examples.

### INDUSTRIAL APPLICABILITY

The oil-in-water emulsion of the present invention has excellent storage stability, a less squeaky feeling, and a good texture when used, and therefore can be used in cosmetics and quasi-drugs. Furthermore, since the oil-in-water emulsion of the present invention has excellent storage stability, it can be suitably used in the fields of paints, cleaning agents, foods, building materials, paper, fibers, ceramics, toiletries, sanitary products, and drainage.

1. A solid emulsifier for oil-in-water emulsions, the solid emulsifier comprising
metal oxide particles having a volume average particle size of 0.2 to 0.4 μm, a specific surface area of 5 to 20 m²/g, and a hydrophobicity of 5 to 35, with the hydrophobicity being measured by the following method:
<Method of Measuring Hydrophobicity>
(1) 50 mL of ion-exchanged water is placed in a 100 mL beaker;
(2) 0.2 g of the metal oxide particles as a measurement sample is added to the beaker in the step (1), followed by stirring with a PTFE stirring bar with a length of 30 mm and a diameter of 8 mm, wherein a stirring speed is adjusted so that when a stirring state in the beaker is observed from a side of the beaker, a lowest point of a vortex created by the stirring is positioned above the midpoint of a distance from a bottom of the beaker to a liquid surface;
(3) 0.5 mL of methanol is added dropwise using a burette with the stirring continued, the stirring is stopped after 30 seconds from the dropwise addition, and 30 seconds after stopping the stirring, the presence or absence of the metal oxide particles at the liquid surface is visually checked;
(4) the step (3) is repeated until the metal oxide particles can no longer be visually observed at the liquid surface; and
(5) a percentage of a volume (mL) of the methanol dropwise added in the steps (3) and (4) relative to a sum of a volume (50 mL) of the ion-exchanged water and the volume of the methanol dropwise added in the steps (3) and (4) in the beaker is calculated to determine the hydrophobicity.

2. The solid emulsifier for oil-in-water emulsions according to claim 1,
wherein the metal oxide particles are silica particles.

3. A method for producing an oil-in-water emulsion containing an aqueous phase component and an oil phase component dispersed in the aqueous phase component, the method comprising:
a dispersion step including dispersing the solid emulsifier for oil-in-water emulsions according to claim 1 in an aqueous solvent to obtain an aqueous dispersion; and
an emulsification step including adding the oil phase component to the aqueous dispersion while stirring the aqueous dispersion.

4. The method for producing an oil-in-water emulsion according to claim 3,
wherein the aqueous dispersion contains an alcohol having a water solubility of 500 g/L or higher at 20° C., a mass ratio of the alcohol in the aqueous dispersion is 5 to 55 parts by mass relative to 100 parts by mass of water, and the aqueous dispersion has a pH of 7 to 14 at 25° C.

5. A method for producing an oil-in-water emulsion containing an aqueous phase component and an oil phase component dispersed in the aqueous phase component, the method comprising:
a dispersion step including dispersing the solid emulsifier for oil-in-water emulsions according to claim 1 in an oily component to obtain an oil dispersion; and
an emulsification step including adding the aqueous phase component to the oil dispersion while stirring the oil dispersion.

6. An oil-in-water emulsion comprising the solid emulsifier for oil-in-water emulsions according to claim 1.

7. A cosmetic composition comprising the oil-in-water emulsion according to claim 6.
